(19) United States
(12) Patent Application Publication
(10) Pub. No.: US 2008/0234779 A1
(43) Pub. Date: Sep. 25, 2008

(54) MINIMALLY INVASIVE INTRAOPERATIVE MODULATION OF PATIENT PARAMETERS USING BAROREFLEX ACTIVATION

(75) Inventors: Brad Pedersen, Minneapolis, MN (US); Robert Kieval, Medina, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: CVRX, INC.

(21) Appl. No.: 12/052,568

(22) Filed: Mar. 20, 2008

Related U.S. Application Data
(60) Provisional application No. 60/895,909, filed on Mar. 20, 2007.

(51) Int. Cl. A61N 1/36

(52) U.S. Cl. 607/44

(57) ABSTRACT

A baroreflex therapy system for providing intraoperative patient treatment. The system comprises an internal activation device adapted to be inserted into the throat area of a patient, an external activation device adapted to be located on the outside of a body of a patient such that an anatomical structure within the patient capable of creating a baroreflex response is located between the internal activation device and the external activation device, and an external controller, including a pulse generator operably connected to the internal activation device and the external activation device to deliver baroreflex therapy between the internal activation device and the external activation device.

### RELATED APPLICATIONS

This application claims the benefit of U.S. Provisional Application No. 60/895,909, filed Mar. 20, 2007, the disclosure of which is hereby incorporated by reference in its entirety.

### FIELD OF THE INVENTION

The present invention relates generally to intraoperative patient care. More specifically, the present invention relates to the minimally-invasive modulation of certain patient parameters during surgery by activation of a patient's baroreflex system that may be used with or without pharmacological agents.

### BACKGROUND OF THE INVENTION

Intraoperative care of a patient is an essential part of surgery. As part of intraoperative care, numerous patient conditions are monitored by the surgery team. Vital signs, heart rate, blood oxygenation levels, and blood pressure are examples of patient conditions that may be the subject of intraoperative monitoring. Prompt detection of abnormal patient conditions during surgery is essential for the patient's safety and success of the surgery.

Control of patient parameters during surgery may be accomplished by a variety of methods such as drug therapy, electrical stimulation, or other methods. Drug therapy may be used for intraoperative control of patient parameters, including blood pressure. However, drugs remain in the body for a predetermined amount of time, and in most cases their effects cannot be reversed instantaneously. Intraoperative electrical control of a patient's heart is disclosed in U.S. Pat. No. 6,912,419 to Hill et al. However, the device and method of Hill require a separate invasive surgery for implantation, further adding to the list of possible complications during surgery.

Numerous other devices and methods exist for stimulating, modulating, or controlling parameters in a patient by electrical stimulation, such as blood pressure, heart rate, or nervous system activity. While most electrical stimulation systems utilize chronically implanted electrode arrangements, catheter based temporary pacing and defibrillation leads are also known as described, for example, in U.S. Pat. Nos. 3,769,984, 4,214,594 and 4,357,947.

Devices for monitoring patient parameters during surgery are also known, such as esophageal monitoring of blood flow as described, for example, in U.S. Pat. No. 4,836,214, or esophageal monitoring of cardiac functions as described, for example, in U.S. Pat. No. 6,438,400.

Attempts have been made to provide cardiac stimulation in the form of pacing and defibrillation utilizing esophageal electrodes, as described, for example, in U.S. Pat. No. 5,387,232. However, such efforts have had limited success due to the need for large amounts of electrical energy being applied relatively distant to the heart that can result in burning or other tissue damage to the throat.

Prompt, precise control of blood pressure, heart rate, and other parameters during surgery is often critical. Therefore, it would be desirable to provide an intraoperative control device and method for temporarily providing control of important patient physiological parameters during surgery that is non-invasive and does not require, or can reduce the need for, the use of drugs or other pharmacological agents.

### SUMMARY OF THE INVENTION

The present invention is directed to intraoperative methods and systems for controlling or modulating one or more patient parameters during surgery by activation of a patient's baroreflex system. In one embodiment, one or more electrode structures are removably placed inside the upper body of a patient during a surgical procedure, and one or more electrode structures are placed on the exterior of the patient, generally in alignment with the electrode structures inside the patient. In another embodiment, two or more electrode structures are removably placed inside the upper body of a patient during a surgical procedure with no electrode structures on the exterior of the patient. A control system is connected to the electrode structures, and generates a signal to activate the electrodes. Proper placement of the electrode structures results in activation of a patient's baroreflex system to modulate one or more parameters of the patient.

In one embodiment, the present invention comprises a baroreflex therapy system for providing intraoperative patient treatment. The system comprises an internal activation device adapted to be inserted into the throat area of a patient, an external activation device adapted to be located on the outside of a body of a patient such that an anatomical structure within the patient capable of creating a baroreflex response is located between the internal activation device and the external activation device, and an external controller, including a pulse generator operably connected to the internal activation device and the external activation device to deliver baroreflex therapy between the internal activation device and the external activation device.

In another embodiment, the present invention comprises a method of treating a patient in connection with a surgery. The method comprises providing an internal activation device, providing an external activation device, providing an external controller including a pulse generator, the controller coupled to the internal activation device and the external activation device, and providing instructions. The instructions include inserting the internal activation device into the throat area of the patient prior to surgery, positioning the external activation device on the outside of the body of a patient such that an anatomical structure capable of stimulating the baroreflex of the patient is between the internal activation device and the external activation device, activating, deactivating, or otherwise modulating the internal activation device and/or the external activation device with the controller to effect a change in the baroreflex system of a patient during surgery, and removing the internal activation device and the external activation device after surgery.

In a further embodiment, the present invention comprises a method of modulating a patient parameter during a surgical procedure. The method comprises providing a baroreflex therapy system, including an internal activation device, an external activation device, and an external controller including a pulse generator, the controller coupled to the internal activation device and the external activation device. The method further comprises providing instructions for operating the baroreflex therapy system, including inserting the internal activation device into the throat area of the patient, positioning the external activation device on the outside of the body of a patient such that an anatomical structure capable of creating a baroreflex response of the patient is between the internal activation device and the external activation device, establishing a target range of a patient physiological parameter associated with a period of time relative to the surgical procedure, and activating, deactivating, or otherwise modulating the internal activation device and/or the external activation device with the controller to effect a change in the baroreflex system of a patient in accordance with the target range of the patient physiologic parameter.

### BRIEF DESCRIPTION OF THE DRAWINGS

The invention may be more completely understood in consideration of the following detailed description of various embodiments of the invention in connection with the accompanying drawings, in which:

FIG. 1 is a schematic illustration of the upper torso of a human body showing the major arteries and veins and associated anatomy.

FIG. 2A is a cross-sectional schematic illustration of the carotid sinus and baroreceptors within the vascular wall.

FIG. 2B is a schematic illustration of baroreceptors within the vascular wall and the baroreflex system.

FIG. 3A is a cross-sectional transverse view of a human neck at the sixth cervical vertebra, the neck being split along the mid-sagittal plane.

FIG. 3B is a cross-sectional view along the sagittal plane of a human neck.

FIG. 3C is a schematic cross-sectional transverse view of a human neck.

FIG. 4 is a partial cross-sectional view of the throat region of a human with an airway management device inserted therein.

FIG. 5A is a schematic cross-sectional transverse view of a human neck having an intraoperative modulation device attached thereto according to one embodiment of the present invention.

FIG. 5B is the view of FIG. 5A showing activation of an intraoperative modulation device according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

Various embodiments of the present invention provide devices, systems and methods by which blood pressure, heart rate, respiration, and/or autonomic nervous system activity may be selectively and controllably modulated during surgery via baroreflex activation.

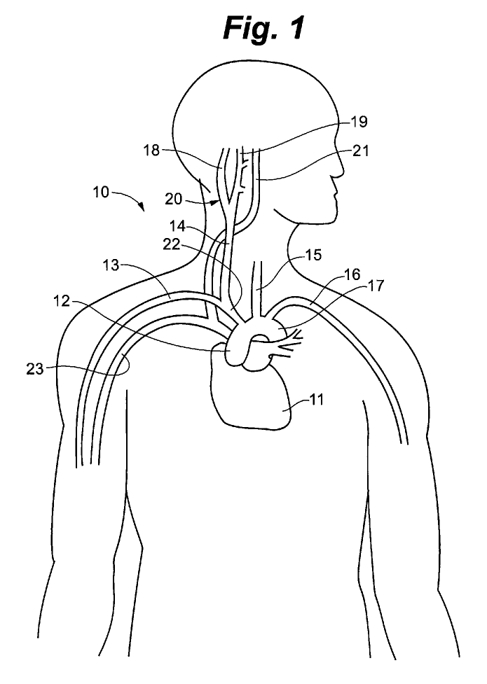

To better understand the present invention, it may be useful to explain some of the basic vascular anatomy associated with the cardiovascular system. FIG. 1 is a schematic illustration of the upper torso of a human body 10 showing some of the major arteries and veins of the cardiovascular system. The left ventricle of the heart 11 pumps oxygenated blood up into the aortic arch 12. The right subclavian artery 13, the right common carotid artery 14, the left common carotid artery 15 and the left subclavian artery 16 branch off the aortic arch 12 proximal of the descending thoracic aorta 17. Although relatively short, a distinct vascular segment referred to as the brachlocephalic artery 22 connects the right subclavian artery 13 and the right common carotid artery 14 to the aortic arch 12. The right carotid artery 14 bifurcates into the right external carotid artery 18 and the right internal carotid artery 19 at the right carotid sinus 20. Although not shown for purposes of clarity only, the left carotid artery 15 similarly bifurcates into the left external carotid artery and the left internal carotid artery at the left carotid sinus.

From the aortic arch 12, oxygenated blood flows into the carotid arteries 18/19 and the subclavian arteries 13/16. From the carotid arteries 18/19, oxygenated blood circulates through the head and cerebral vasculature and oxygen depleted blood returns to the heart 11 by way of the jugular veins, of which only the right internal jugular vein 21 is shown for sake of clarity. From the subclavian arteries 13/16, oxygenated blood circulates through the upper peripheral vasculature and oxygen depleted blood returns to the heart by way of the subclavian veins, of which only the right subclavian vein 23 is shown, also for sake of clarity. The heart 11 pumps the oxygen depleted blood through the pulmonary system where it is re-oxygenated. The re-oxygenated blood returns to the heart 11 which pumps the re-oxygenated blood into the aortic arch as described above, and the cycle repeats.

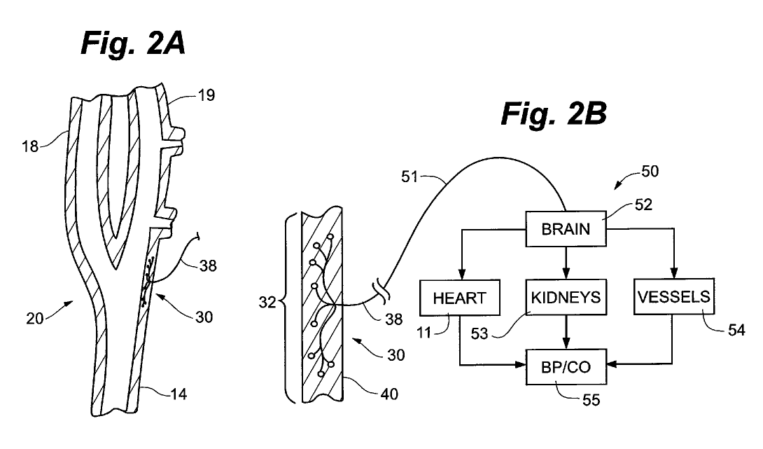

Within the arterial walls of the aortic arch 12, common carotid arteries 14/15 (near the right carotid sinus 20 and left carotid sinus), subclavian arteries 13/16 and brachlocephalic artery 22 there are baroreceptors 30. For example, as best seen in FIG. 2A, baroreceptors 30 reside within the vascular walls of the carotid sinus 20. Baroreceptors 30 are a type of stretch receptor used by the body to sense blood pressure. An increase in blood pressure causes the arterial wall to stretch, and a decrease in blood pressure causes the arterial wall to return to its original size. Such a cycle is repeated with each beat of the heart. Because baroreceptors 30 are located within the arterial wall, they are able to sense deformation of the adjacent tissue, which is indicative of a change in blood pressure. The baroreceptors 30 located in the right carotid sinus 20, the left carotid sinus and the aortic arch 12 play the most significant role in sensing blood pressure that affects the baroreflex system 50, which is described in more detail with reference to FIG. 2B.

With reference now to FIG. 2B, a schematic illustration shows baroreceptors 30 disposed in a generic vascular wall 40 and a schematic flow chart of baroreflex system 50. Baroreceptors 30 are profusely distributed within the arterial walls 40 of the major arteries discussed previously, and generally form an arbor 32. The baroreceptor arbor 32 comprises a plurality of baroreceptors 30, each of which transmits baroreceptor signals to the brain 52 via nerve 38. Baroreceptors 30 are so profusely distributed and arborized within the vascular wall 40 that discrete baroreceptor arbors 32 are not readily discernable. To this end, baroreceptors 30 shown in FIG. 2 are primarily schematic for purposes of illustration and discussion. It will be assumed that baroreceptors 30 are connected to the brain 52 via the nervous system 51, and brain 52 may activate a number of body systems, including the heart 11, kidneys 53, vessels 54, and other organs/tissues via neural and neurohormonal activity.

Baroreceptor signals in the arterial vasculature are used to activate a number of body systems which collectively may be referred to as the baroreflex system. For the purposes of the present invention, it will be assumed that the “receptors” in the venous and cardiopulmonary vasculature and heart chambers function analogously to the baroreceptors in the arterial vasculature, but such assumption is not intended to limit the present invention in any way. In particular, the methods described herein will function and achieve at least some of the stated therapeutic objectives regardless of the precise and actual mechanism responsible for the result. Moreover, the present invention may activate baroreceptors, mechanoreceptors, pressoreceptors, stretch receptors, chemoreceptors, or any other venous, heart, or cardiopulmonary receptors which affect the blood pressure, nervous system activity, and neurohormonal activity in a manner analogous to baroreceptors in the arterial vasculation. For convenience, all such venous receptors will be referred to collectively herein as “baroreceptors” or “receptors” unless otherwise expressly noted.

While there may be small structural or anatomical differences among various receptors in the vasculature, for the purposes of some embodiments of the present invention, activation may be directed at any of these receptors and/or nerves and/or nerve endings from these receptors so long as they provide the desired effects. In particular, such receptors will provide afferent signals, i.e., signals to the brain, which provide the blood pressure and/or volume information to the brain. This allows the brain to cause “reflex” changes in the autonomic nervous system, which in turn modulate organ activity to maintain desired hemodynamics and organ perfusion. Stimulation of the baroreflex system may be accomplished by stimulating such receptors, nerves, nerve fibers, or nerve endings, or any combination thereof.

Various methods, devices, and systems relating to baroreceptor and baroreflex activation are described in U.S. Pat. No. 6,522,926, U.S. Patent Publication Nos. US 2004/0010303, US 2006/0004417, US 2006/0111626, US 2004/0019364, US 2004/0254616, US 2005/0251212, and US 2005/0154418, and U.S. patent application Ser. No. 12/038,707, filed Feb. 27, 2008 entitled “External Baroreflex Activation” and attached hereto as Appendix A, the disclosures of which are hereby incorporated by reference in their entirety. Although activation of the baroreflex system has been the subject of these patent applications and patents assigned to the assignee of the present application, the focus of the present invention is the effect of baroreflex activation during surgery to modulate or otherwise control blood pressure, heart rate, respiration, neurohormonal activity, or other patient parameters or conditions.

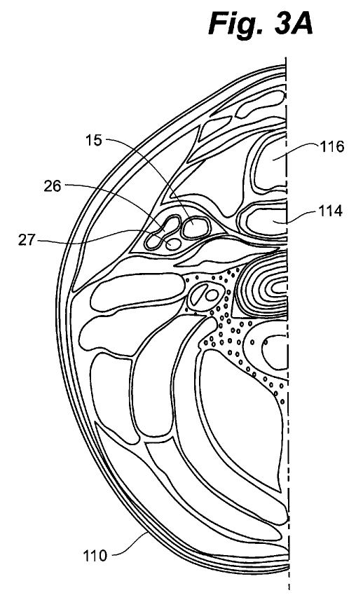

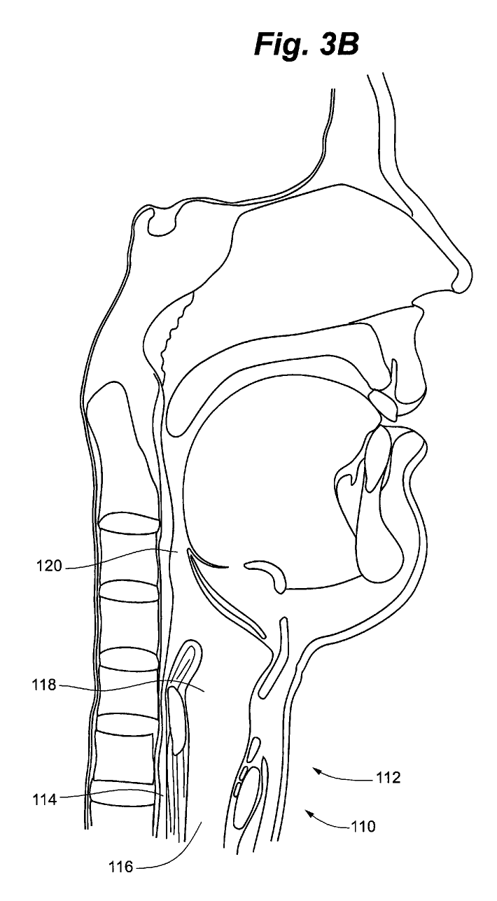

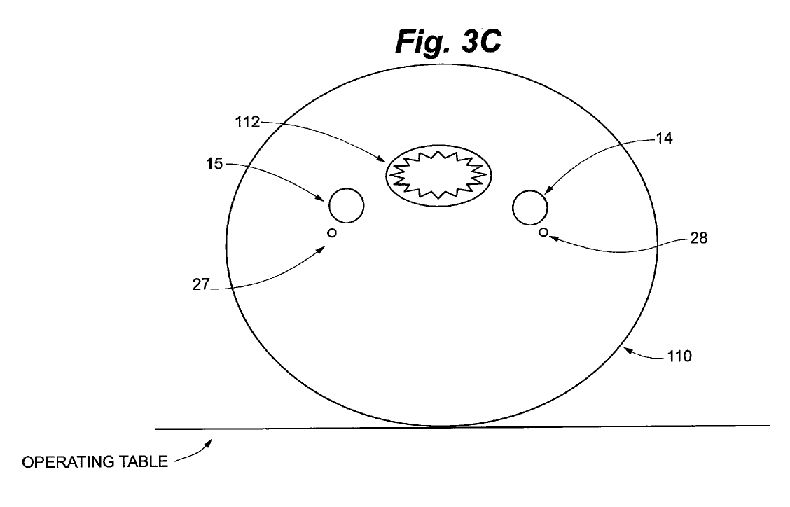

Referring now to FIGS. 3A-3C, various views of a human neck 110 are depicted. Major components of the neck 110 pertinent to the present invention include the throat 112, esophagus 114, trachea 116, larynx 118, pharynx 120 (not pictured), common carotid artery 24/25, internal jugular vein 21/26, aortic arch 12 (not pictured) and vagus nerve 27/28.

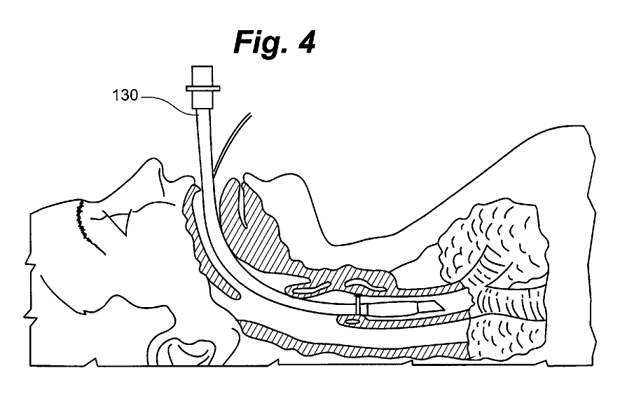

Referring now to FIG. 4, a side cross-sectional view of the neck and head of a human is depicted. During surgery, it may be necessary to provide airway management to a patient, such as with the use of an endotracheal tube 130 inserted in the trachea 116 of the patient. Endotracheal tube 130 may be configured in a variety of ways depending on the desired use, as will be appreciated by one skilled in the art.

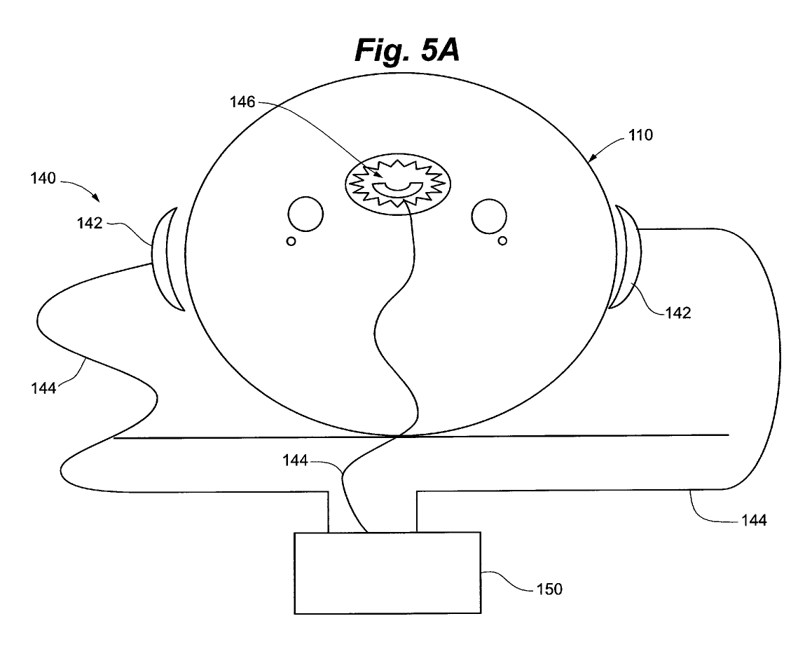

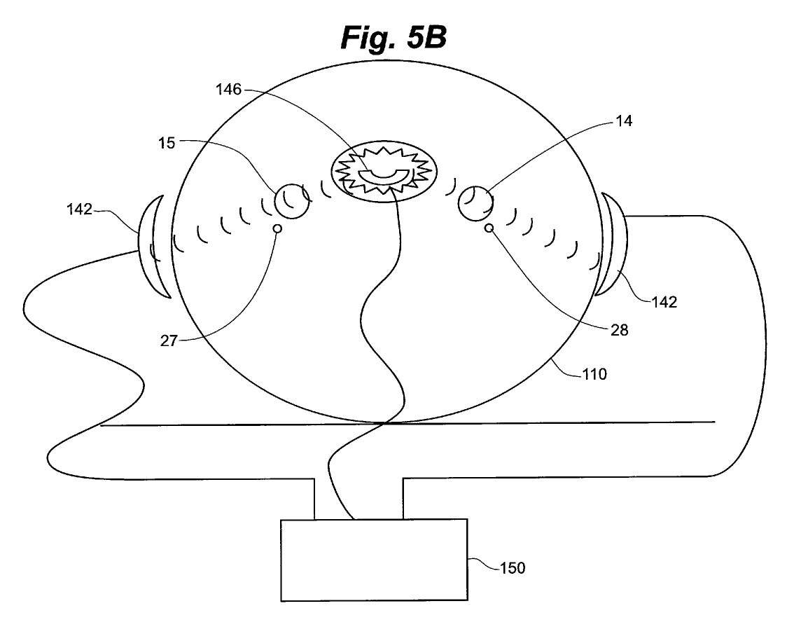

An intraoperative modulation device 140 is schematically depicted in FIG. 5A. In one embodiment, device 140 includes one or more external activation devices 142 coupled via leads 144 to a control system 150. Further, one or more internal activation devices 146 are removably disposed within a patient's body and are coupled to control system 150. In an alternate embodiment, activation devices 142 and/or 146 may be linked to control system 150 by a wireless link, and may also be provided with battery power and a local controller to receive and send sense information and/or delivery therapy. In an alternate embodiment, no external electrode structures 142 are used in conjunction with device 140.

In one embodiment, external activation device 142 may comprise an electrode structure having a generally flexible elastomeric base and one or more electrodes. Internal activation device 146 may similarly comprise an electrode structure having a flexible elastomeric base and one or more electrodes. Electrode structure 146 may be configured in a number of different arrangements depending on the desired application, and may be configured to comprise one or more electrodes secured directly to a structure (discussed further below). Electrode structures 142 and 146 may each comprise a single electrode, an electrode pair, a multiple electrode arrangement, one or more bipolar electrodes, or other arrangement apparent to one skilled in the art or disclosed in the references previously incorporated herein. Additional configurations of electrodes and disclosure regarding multiple electrode configurations is disclosed in U.S. patent application Ser. No. 11/862,508 entitled “Electrode Array Structures and Methods of Use for Cardiovascular Reflex Control,” the disclosure of which is attached hereto as Appendix B and is incorporated herein by reference in its entirety.

In an example embodiment, one or more internal electrode structures 146 are coupled to a structure adapted to be inserted into the throat area of a patient. The structure is configured to be capable of insertion through a patient's nose or mouth. The structure can be inserted into a patient's pharynx, larynx, trachea, or esophagus, depending on the desired application. In a further example embodiment, one or more internal electrode structures 146 are coupled to an airway management device such as endotracheal tube 130. In another example embodiment, one or more internal electrode structures 146 adapted to be located proximate one or more baroreceptor structures in the patient and which are coupled to or placed through other surgical devices, such as catheters, Swan Ganz catheters, Central Venous Access Catheters, Peripherally Inserted Central Catheters (PICC lines), Hickman catheters, Broviac catheters, or Groshong catheters.

Control system 150 may generally include components such as a processor, a memory, a signal generator, one or more sensors, an input device, and a power supply. Control system 150 may also be integrated with other patient monitoring systems commonly used during surgical procedures. Control system 150 may operate in open loop mode utilizing commands from the input device, or in closed loop mode utilizing feedback from the one or more sensors. In closed loop operation, data received from the one or more sensors is used to modify or alter the baroreflex therapy. Control system 150 may also operate in whole or in part based on an algorithm stored in the memory.

Suitable sensors may comprise any suitable device that measures or monitors a parameter (physiologic or otherwise) indicative of the need to modify the activity of one or more patient functions, such as heart rate, baroreflex system, autonomic nervous system, or other nervous system. For example, the sensor may comprise a physiologic transducer or gauge that measures ECG, blood pressure (systolic, diastolic, average or pulse pressure), blood volumetric flow rate, blood flow velocity, blood pH, O2 or CO2 content, pulse rate, mixed venous oxygen saturation (SVO2), vasoactivity, nerve activity, tissue activity, body movement, body temperature, activity levels, respiration, or composition. Examples of suitable transducers or gauges for the sensor include ECG electrodes, a piezoelectric pressure transducer, an ultrasonic flow velocity transducer, an ultrasonic volumetric flow rate transducer, a thermodilution flow velocity transducer, a capacitive pressure transducer, a membrane pH electrode, an optical detector (SVO2), tissue impedance (electrical), a pulse oximetry sensor, or a strain gauge. Multiple sensors of the same or different type at the same or different locations may be utilized.

In one embodiment, one or more electrodes on electrode structures 142, 146 and/or 148 may be used as feedback sensors when not enabled for activation. Alternatively, a separate feedback electrode structure may be provided. The feedback sensor electrode may be used to measure or monitor electrical conduction in the vascular wall to provide data analogous to an ECG. Alternatively, such a feedback sensor electrode may be used to sense a change in impedance due to changes in blood volume during a pulse pressure to provide data indicative of heart rate, blood pressure, or other physiologic parameter.

Control system 150 generates a control signal transmitted to electrode structures 142 and 146. The control signal generated by control system 150 may be continuous, periodic, alternating, episodic or a combination thereof, as dictated by an algorithm contained in the memory. Continuous control signals include a constant pulse, a constant train of pulses, a triggered pulse and a triggered train of pulses. Examples of periodic control signals include each of the continuous control signals described above which have a designated start time (e.g., beginning of each period) and a designated duration (e.g., seconds or minutes) that would occur during the surgical procedure. Examples of alternating control signals include each of the continuous control signals as described above which alternate between right and left output channels, for example.

In electrical activation embodiments wherein the output signal comprises a pulse train, several other signal characteristics may be changed in addition to the pulse characteristics described above. The control or output signal may comprise a pulse train which generally includes a series of pulses occurring in bursts. Pulse train characteristics which may be changed include, but are not limited to: burst amplitude (equal to pulse amplitude if constant within a burst packet), burst waveform (i.e., pulse amplitude variation within burst packet), burst frequency (BF), and burst width or duration (BW). The signal or a portion thereof (e.g., burst within the pulse train) may be triggered by any of the events discussed previously, or by a particular portion of an arterial pressure signal or an ECG signal (e.g., R wave, or phase of respiration, etc), or another physiologic timing indicator. If the signal or a portion thereof is triggered, the triggering event may be changed and/or the delay from the triggering event may be changed.

Additional information relating to suitable control systems applicable to the present invention can be found in any of the disclosures already incorporated by reference herein.

Turning now to the use of intraoperative modulation device 140 as shown, for example, in FIG. 5B, one or more internal electrode structures 146 are inserted into a patient, such that electrode structures 146 are located proximate a location suitable for generating a baroreflex response. Such locations may include anatomical structures such as aortic arch 12, carotid arteries 14/15, carotid sinus 20, internal jugular veins 21/26, pulmonary artery (not pictured) and vagus nerves 27/28. In one embodiment, internal electrode structure 146 may be inserted into esophagus 114 or trachea 116, which are routed proximate carotid arteries 14/15, internal jugular veins 21/26, pulmonary artery (not pictured) and aortic arch 12. In another embodiment, internal electrode structure 146 may be inserted into larynx 118, which is routed proximate internal jugular veins 21/26, carotid sinus 20, and vagus nerves 27/28.

One or more external electrode structures 142 are positioned on the outside of the patient's body. The placement of external electrode structures 142 is such that an anatomical structure capable of creating a baroreflex response in the patient is located between external electrode structures 142 and internal electrode structures 146. In one example embodiment, if an internal electrode structure 146 is located in larynx 118 or trachea 120, external electrode structures 142 will be placed on the outside of a patient's neck 110, such as depicted in FIG. 5A, so as to activate the patient's baroreflex system. A conductive gel may be applied to external electrode structure 142 so as to decrease electrical resistance during activation.

In another example embodiment, an internal electrode structure 146 is located lower in trachea 120 or esophagus 114, and external electrode structures 142 may be placed on the lower neck upper back, or upper torso to be aligned with internal electrode structure 146 so as to activate the patient's baroreflex system.

In another example embodiment, an internal electrode structure 146 is positioned in trachea 120 or esophagus 114, and another internal electrode structure 148 is introduced by a temporary catheter into a vein, for example, in the neck and then positioned proximate the internal electrode structure 146.

Control system 150 is operatively coupled to electrode structures 142, 146, or 146, 148 and provides activation (or control) signals to the electrode structures. Electrode structures 142, 146 or 146, 148 comprise an anode/cathode pair or cathode/anode pair. In the case of multiple electrodes or multiple electrode structures, control signals may be configured to activated electrodes or electrode structures individually or in combinations.

Electrical activation of electrode structures 142, 146 or 146, 148 causes electrical stimulation of tissues located therebetween. Each electrode structure is positioned so as to locate tissues capable of modifying the patient's baroreflex system in the electrical field created between external electrode structure 142 and internal electrode structure 146 or between internal electrode structures 146 and 148.

In another example embodiment, intraoperative modulation device 140 comprises solely an internal electrode structure 146. No external electrode structures 142 are used. In such an embodiment, the internal electrodes 146 are positioned so as to locate tissues capable of modifying the patient's baroreflex system in the electrical field created by electrical activation of electrodes 146. In such an embodiment, the electrical activation of electrodes 146 will be at a higher level as compared to alternate embodiments utilizing external electrode structures.

In another mode of operation, the present invention can be used to adjust or modulate one or more patient physiological parameters during surgery. For example, it may be desirable during certain surgical procedures to lower the blood pressure of a patient, such as when the surgery involves accessing or repairing major blood vessels such as veins and arteries. To lower blood pressure of the patient to facilitate procedures performed on or in blood vessels, control system 150 transmits a signal to electrode structures 142 and/or 146, which activate the baroreflex response of the patient.

In another embodiment, it may be desirable during certain surgical procedures to raise the blood pressure of a patient. Control system 150 may be operated to transmit a signal to electrode structures 142 and/or 146 to inhibit the baroreflex response of the patient. Baroreflex inhibition may be accomplished in one embodiment by high frequency activation signals.

In another embodiment, it may be desirable to control the patient physiological parameters such as blood pressure, heart rate, and/or respiration of a patient during surgery, and the present invention may be used to activate or inhibit the baroreflex response of the patient in order to maintain the patient physiological parameter at a desired level or within a desired range. Control system 150 may be configured to raise, lower, maintain or otherwise control one or more patient physiological parameters during different times of the same surgical procedure. Control system 150 may receive feedback from one or more sensors, or be operated manually by a surgeon or assistant.

Further, the present invention may be used in conjunction with operative drugs administered to the patient during surgery, to enhance or counteract any effects of operative drugs such as anesthesia.

In addition to electrical stimulation of the baroreflex system, it is possible that changing the electrical potential across the tissue surrounding baroreceptors 30 may cause the surrounding tissue to stretch or otherwise deform, thus mechanically activating baroreceptors 30.

In an example embodiment, the waveform of the activation signal is selected so as to not initiate an unfavorable physiologic response in the patient, such as for example, in the muscular system, nervous system, or other body system.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

1. A baroreflex therapy system for providing intraoperative patient treatment, comprising:
an internal activation device adapted to be inserted into the throat area of a patient;
an external activation device adapted to be located on the outside of a body of a patient such that an anatomical structure within the patient capable of creating a baroreflex response is located between the internal activation device and the external activation device; and
an external controller, including a pulse generator operably connected to the internal activation device and the external activation device to deliver baroreflex therapy between the internal activation device and the external activation device.

2. The system of claim 1, wherein the internal activation device and the external activation device each include at least one electrode and the baroreflex therapy comprises electrical pulses.

3. The system of claim 2, wherein the internal activation device and the external activation device are further configured to be operated as sensors adapted to sense a physiological patient parameter and transmit a signal to the controller, the signal indicative of the physiological patient parameter.

4. The system of claim 2, further comprising a sensor separate from the internal activation device and the external activation device adapted to sense a physiological patient parameter and transmit a signal to the controller, the signal indicative of the physiological patient parameter.

5. The system of claims 3 or 4, wherein the controller is adapted to activate, deactivate or otherwise modulate the internal activation device and/or the external activation device based on a signal received from the sensor.

6. The system of claim 1 wherein the internal activation device is coupled to an airway management device.

7. The system of claim 1 wherein the internal activation device is coupled to a catheter.

8. A method of treating a patient in connection with a surgery, comprising:
providing an internal activation device;
providing an external activation device;
providing an external controller including a pulse generator, the controller coupled to the internal activation device and the external activation device;
providing instructions, including:
inserting the internal activation device into the throat area of the patient prior to surgery;
positioning the external activation device on the outside of the body of a patient such that an anatomical structure capable of creating a baroreflex response of the patient is between the internal activation device and the external activation device;
activating, deactivating, or otherwise modulating the internal activation device and/or the external activation device with the controller to effect a change in the baroreflex system of a patient during surgery; and
removing the internal activation device and the external activation device after surgery.

9. The method of claim 8, further comprising:
providing a sensor;
providing instructions including:
generating a sensor signal indicative of a patient physiological parameter using the sensor; and
activating, deactivating, or otherwise modulating the at least one electrode with the controller as a function of the sensor signal.

10. The method of claim 9, wherein the sensor is selected from the group consisting of the internal activation device and the external activation device.

11. The method of claim 8, wherein inserting the internal activation device into the throat area of the patient comprises inserting the internal activation device into a location selected from the group consisting of the pharynx, the larynx, the trachea, and the esophagus.

12. A method of modulating a patient parameter during a surgical procedure, comprising:
providing a baroreflex therapy system, including:
an internal activation device;
an external activation device; and
an external controller including a pulse generator, the controller coupled to the internal activation device and the external activation device;
providing instructions for operating the baroreflex therapy system, comprising:
inserting the internal activation device into the throat area of the patient;
positioning the external activation device on the outside of the body of a patient such that an anatomical structure capable of creating a baroreflex response of the patient is between the internal activation device and the external activation device;
establishing a target range of a patient physiological parameter associated with a period of time relative to the surgical procedure; and
activating, deactivating, or otherwise modulating the internal activation device and/or the external activation device with the controller to effect a change in the baroreflex system of a patient in accordance with the target range of the patient physiologic parameter.

13. The method of claim 12, wherein the period of time relative to the surgical procedure is during an administration of anesthesia, and the target range of the patient physiological parameter is set to maintain blood pressure at a range prior to the administration of anesthesia.

14. The method of claim 12, wherein the period of time relative to the surgical procedure is during an access of a major blood vessel and the target range of the patient physiological parameter is set to lower blood pressure relative to a range prior to the access of a major blood vessel.

15. The method of claim 12, wherein the period of time relative to the surgical procedure is proximate the end of the surgical procedure and the target range of the patient physiological parameter is set to increase blood pressure relative to blood pressure during the surgical procedure.
